(19) United States
(12) Patent Application Publication
(10) Pub. No.: US 2026/0061222 A1
(43) Pub. Date: Mar. 5, 2026

(54) RADIATION THERAPY SYSTEM, RADIATION THERAPY METHOD AND PROGRAM

(71) Applicants: TOSHIBA ENERGY SYSTEMS & SOLUTIONS CORPORATION, Kawasaki-shi (JP); KANAGAWA PREFECTURAL HOSPITAL ORGANIZATION, Yokohama-shi (JP)

(72) Inventors: Yohsuke KUSANO, Ayase (JP); Toshikatsu OOTSUKI, Kawasaki (JP); Akihiko NAKAMURA, Yokohama (JP)

(73) Assignees: TOSHIBA ENERGY SYSTEMS & SOLUTIONS CORPORATION, Kawasaki-shi (JP); KANAGAWA PREFECTURAL HOSPITAL ORGANIZATION, Yokohama-shi (JP)

(21) Appl. No.: 19/279,045

(22) Filed: Jul. 24, 2025

(30) Foreign Application Priority Data
Aug. 29, 2024 (JP) 2024-147800

(51) Int. Cl. A61N 5/10; G16H 20/40

(52) U.S. Cl. CPC A61N 5/1049; G16H 20/40; A61N 2005/1061

(57) ABSTRACT

A radiation therapy system includes a holding unit configured to hold first movement information for moving a tabletop such that a first position of a patient's focus identified by first voxel data matches an isocenter in a dose of a beam administered to the patient's focus, and an irradiation condition of the beam, which is set to maximize a focal coverage rate of the patient's focus irradiated with the beam at the first position; an identification unit configured to identify a second position of the patient's focus by second voxel data imaged immediately before emitting the beam; and an update unit configured to update the first movement information with second movement information to maximize the focal coverage rate at the position of the patient's focus without changing the irradiation condition.

### CROSS-REFERENCE TO RELATED APPLICATIONS

This application is based upon and claims the benefit of priority from Japanese Patent Application No. 2024-147800 filed on Aug. 29, 2024, the entire contents of which are incorporated herein by reference.

### BACKGROUND OF THE INVENTION

### Field of the Invention

The present invention embodiment relates to a radiation therapy technique for treating patient's focus by using beam irradiation.

### Description of the Related Art

Radiation therapy is a treatment technique for destroying focuses (e.g., cancers) in patients via beam irradiation. Particle beams, such as carbon ion beams, may decay in kinetic energy when passing through the patient's body, and then particle beams would be rapidly stopped when velocity drops to a certain value. Particle beams form a dose of beam distribution referred to as the Bragg peak near the stop point thereof to release energy.

The irradiation condition and the patient's position should be determined such that a patient's focus will be positionally adjusted to the Bragg peak corresponding to the terminal position in the range of beams releasing energy. Thus, it is possible to realize treatment to maximize an absorbed dose of beams irradiated to focal tissues while keeping an absorbed dose of beams irradiated to normal tissues to a minimum.

For this reason, inaccurate positional adjustment of the Bragg peak to a patient's focus might result in a fear that normal tissues will be destroyed via beam irradiation. Therefore, treatment plans should be made before treatment administration for irradiating beams to patients. In the treatment plan, a CT scan is performed on a patient to obtain voxel data, thus three-dimensionally identifying the shape and the position of a patient's focus in the body. The setting position of a tabletop for fixing a patient's body, and the irradiation condition should be determined to apply an adequate dose of beams to focal tissues while reducing the dose of beams irradiated to normal tissues as small as possible.

It may take a certain time to determine the setting position of a tabletop, and the irradiation condition described above. In other words, it may generally take several days after a treatment plan before treatment administration. Internal organs such as a pancreas surrounded by digestive tracts in the body are characterized in that the position thereof in the body cannot be fixed to a certain position according to the amount of food ingested by a patient and the movement of a patient's body. The patient's focus appearing in any internal organ may be displaced in the body in each stage of treatment planning and treatment administration.

In this case, the interior of a patient's body shall be imaged even in the stage of treatment administration, thus observing a patient's focus displaced after the stage of treatment planning. The beam irradiation will be executed after correcting the setting position of the tabletop, and the irradiation condition based on the observed displacement of a patient's focus.

Patent Document 1 (Japanese Patent Application Publication No. 2023-172165) discloses a radiation therapy system simplifying the movement of a treatment table when acquiring 3D images. Patent Document 2 (Japanese Patent Application Publication No. 2019-147029) discloses a particle beam treatment system capable of accurately irradiating particle beams to the affected area of a patient regardless of a treatment plan. Patent Document 3 (Japanese Patent Application Publication No. 2016-209012) discloses a particle beam treatment system capable of accurately irradiating particle beams to the affected area of a patient regardless of a treatment plan.

The attenuation of beams passing through a patient's body is greatly influenced by the density of internal tissues and intestinal gas in the beam range. Thus, any discrepancy in terms of water equivalent lengths towards the patient's focus assumed in the stage of treatment planning may lead to any difference in the beam range inside a patient's body rather than the beam range assumed in an initial treatment plan.

The above discrepancy may bring the Bragg peak to be formed at a different position than the actual focal position imaged in the stage of treatment administration. Therefore, simply changing the setting position of a tabletop based on the positional displacement of patient's bones or focuses checked via an X-ray scan may lead to an insufficient dose of beams administered to focal tissues, which would possibly increase the dose of beams irradiated to normal tissues.

When correcting the irradiation condition to prevent an increasing dose of beams irradiated to normal tissues, it takes a long time for correction. This may impose a burden on a patient and increase the time of treatment.

The present invention embodiment is made in consideration of the above circumstances, and aims to provide a radiation therapy technique for maximizing an absorbed dose of beams irradiated to focal tissues in a short period of time while keeping an absorbed dose of beams irradiated to normal tissues to a minimum, even when the stage of treatment planning is affected by any change in the position of a patient's focus in the body or any change in the density of internal tissues or intestinal gas in the beam range.

### SUMMARY OF THE INVENTION

According to the present invention embodiment, a radiation therapy system includes a hold unit configured to hold first movement information for moving a tabletop used to fix a patient's body thereon such that a first position of a patient's focus identified by first voxel data imaging the interior of the patient's body matches an isocenter in a dose of a beam administered to the patient's fucus, and the irradiation condition, which is set to maximize a focal coverage rate of a patient's focus irradiated with the beam at the first position; an identification unit configured to identify a second position of the patient's focus by second voxel data imaging the interior of the patient's body immediately before emitting the beam; and an update unit configured to update the first movement information with second movement information to maximize the focal coverage rate at the position of the patient's focus without changing the irradiation condition.

According to the present invention embodiment, a radiation therapy method includes the steps of: holding first movement information for moving a tabletop used to fix a patient's body thereon such that a first position of a patient's focus identified by first voxel data imaging the interior of the patient's body matches an isocenter in a dose of a beam administered to the patient's focus; holding an irradiation condition, which is set to maximize a focal coverage rate of the patient's focus irradiated with the beam at the first position; identifying a second position of the patient's focus by second voxel data imaging the interior of the patient's body immediately before emitting the beam; and updating the first movement information with second movement information to maximize the focal coverage rate at the position of the patient's focus without changing the irradiation condition.

### Effect of the Invention

According to the present invention embodiment, it is possible to maximize an absorbed dose of beams irradiated to focal tissues in a short period of time while keeping an absorbed dose of beams irradiated to normal tissues to a minimum, even when the stage of treatment planning is affected by any change in the position of a patient's focus in the body or any change in the density of internal tissues or intestinal gas in the beam range.

### BRIEF DESCRIPTION OF THE DRAWINGS

FIG. 1 is a configuration diagram of a radiation therapy system according to the first embodiment of the present invention.

FIG. 2 is an explanatory schematic of a rotational motion around an isocenter of a rotating gantry of the radiation therapy system according to the present invention embodiment.

FIG. 3 is a configuration diagram of an update unit of the radiation therapy system according to the second embodiment of the present invention.

FIG. 4A is a graph showing a dose of beam distribution in a propagating direction of beams irradiated to a patient's focus at the first position of first voxel data imaged in the stage of treatment planning.

FIG. 4B is a graph showing a dose of beam distribution in a propagating direction of beams irradiated to a patient's focus at the second position of second voxel data imaged in the stage of treatment administration.

FIG. 4C is a graph showing a dose of beam distribution in a propagating direction of beams irradiated to a patient's focus at the selected position which is selected such that the focal coverage rate will have a maximum value by updating the position of an isocenter.

FIG. 5A is a flowchart showing the processing related to treatment planning in the radiation therapy system according to the present invention embodiment.

FIG. 5B is a flowchart showing the processing related to treatment administration of the radiation therapy system according to the present invention embodiment.

### DETAILED DESCRIPTION OF THE INVENTION

### First Embodiment

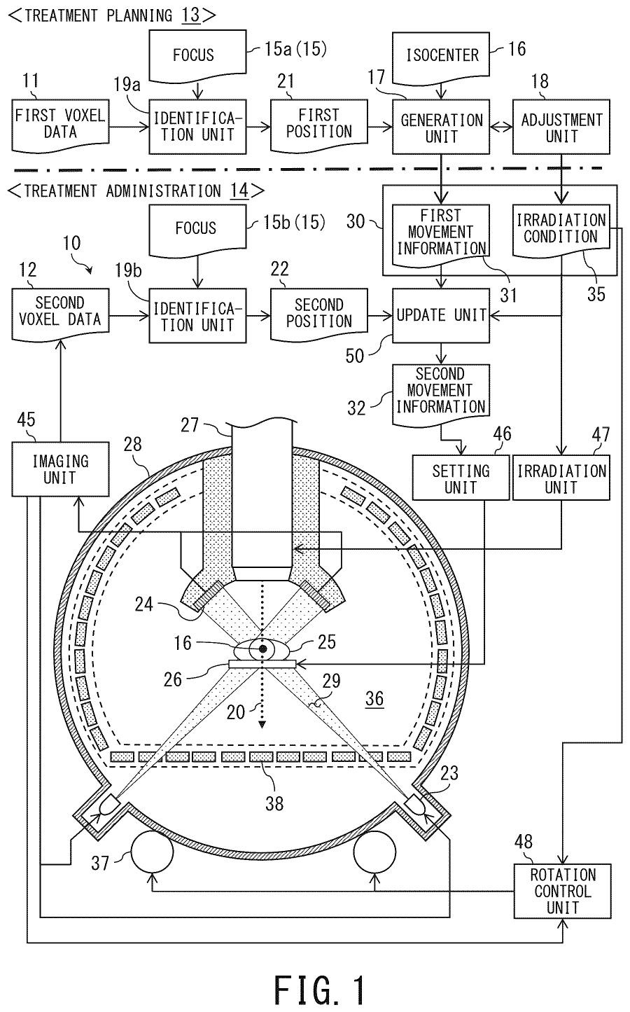

Hereinafter, the present invention embodiments will be described with reference to the accompanying drawings. FIG. 1 is a configuration diagram of a radiation therapy system 10 according to the first embodiment of the present invention. Treatment planning 13 is carried out in advance prior to treatment administration 14 using the radiation therapy system 10. The treatment planning 13 is carried out in a different place than treatment chamber 36 used to implement the treatment administration 14. In the treatment planning 13, a patient 25 is fixed to a tabletop 26 in the same posture as the posture to receive a beam 20 irradiated thereto in the treatment administration 14, thus imaging first voxel data 11. Finally, the treatment planning 13 will produce first movement information 31 and irradiation condition 35.

The first voxel data 11 acquired in the stage of the treatment planning 13 represents a three-dimensional image (or a stereoscopic image), imaging the interior body of the patient 25, which is obtained by stereoscopic imaging equipment, such as X-ray CT (Computed Tomography) or nuclear magnetic resonance (MRI: Magnetic Resonance Imaging).

The first movement information 31 is data for moving the tabletop 26 used to fix the patient 25 thereon such that a first position 21 of a focus 15a (15), which is identified by the first voxel data 11 imaging the interior body of the patient 25, matches an isocenter 16 of the beam 20 for administering a dose of beams to the focus 15a.

An identification unit 19a (19) is configured to identify the first position 21 of the focus 15 distinct from normal tissues in a coordinate system referring to an area showing no positional displacement of bones in the first voxel data 11 or a reference position of imaging equipment.

A generation unit 17 has a function of generating movement information input to a transport mechanism (not shown) for positioning the tabletop 26 in the treatment chamber 36. In the stage of the treatment planning 13, the generation unit 17 generates the first movement information 31 as a command given to the transport mechanism (not shown) for positioning the tabletop 26 such that the first position 21 of the focus 15a matches the isocenter 16 of the beam 20.

The transport mechanism (not shown) has a unique function to adjust the position and the orientation of the tabletop 26 in a coordinate system referring to the treatment chamber 36. The generation unit 17 is configured to convert the first position 21 defined in the coordinate system of the first voxel data 11 to the coordinate system of the treatment chamber 36, thereby generating the first movement information 31 such that the first position 21 matches the isocenter 16 originally defined in the coordinate system of the treatment chamber 36.

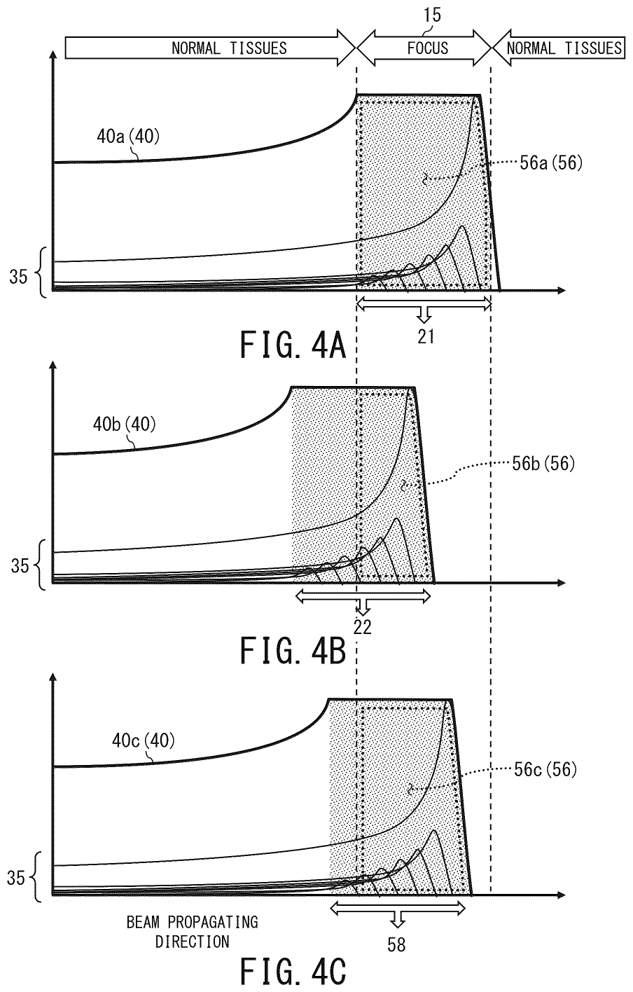

An adjustment unit 18 is configured to adjust the irradiation condition 35 of the beam 20 irradiated to the focus 15, such as a dose of irradiation, an irradiation angle, an irradiation range, and the number of times for irradiating beams. In the treatment planning 13, the irradiation condition 35 is set to maximize a focal coverage rate 56a (see FIGS. 4A to 4C) of the patient 25 irradiated with the beam 20 at the first position 21. The focal coverage rate 56 refers to a focal coverage rate of the focus 15 receiving a prescribed dose of irradiation.

The radiation therapy system 10 in use of the operation of the treatment administration 14 includes a holding unit 30 configured to hold the irradiation condition 35 of the beam 20 and the first movement information 31 of the tabletop 26, an identification unit 19b (19) configured to identify a second position 22 of the focus 15 by second voxel data 12 imaging the interior body of the patient 25 immediately before irradiation of the beam 20, and an update unit 50 configured to update the first movement information 31 with second movement information 32 such that the focal coverage rate 56 will be maximized at the position of the focus 15 of the patient 25 without changing the irradiation condition 35.

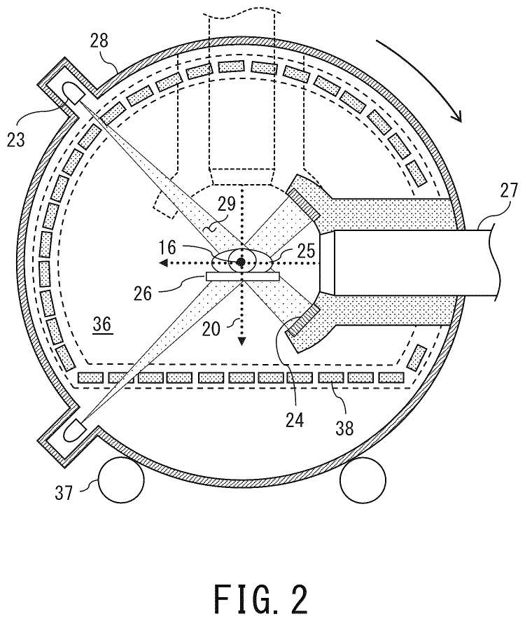

FIG. 2 is an explanatory schematic of a rotational motion about the isocenter 16 of a rotating gantry 28 in the radiation therapy system 10 according to the first embodiment of the present invention. An irradiation port 27 used to irradiate the beam 20 is fixed to the rotating gantry 28 of the radiation therapy system 10. Since the irradiation port 27 rotates about a rotation axis together with the rotating gantry 28, it is possible to irradiate the beam 20 to the focus 15 (15a, 15b) of the patient 25 in an arbitrary direction without tilting the tabletop 26. By rotating the irradiation port 27 around the body axis of the patient 25 in this way, it is possible to irradiate the beam 20 to the focus 15 in multiple directions, thus suppressing the dose of beams irradiated to normal tissues around the focus 15 to a minimum.

The rotating gantry 28 generally constituted of a large-size structure having a cylindrical shape is designed to rotate about the rotation axis by rotationally driving a plurality of rotation drive parts 37 circumscribed on the outer circumference at both edges thereof. The treatment chamber 36 is formed inside a moving platform 38, which is disposed along the inner circumference of the rotating gantry 28 and circumferentially rotates with the rotation of the rotating gantry 28. The weight of the rotating gantry 28 is supported by a stationary system (not shown) via the rotation drive parts 37.

In addition to the irradiation port 27, the rotating gantry 28 is equipped with a plurality of beam transport ducts, beam deflection magnets, and other control devices and structures, the illustrations of which re omitted here. The beam 20 is generated by accelerating ions (heavy particles or proton ions) produced by an ion source by a linear accelerator whose illustration is omitted here, and injecting ions into a circular accelerator (not shown) to raise the energy of ions to the preset energy level. The beam 20 picked up from the circular accelerator is transported by a beam transport system (not shown), and then the beam 20 is irradiated from the irradiation port 27 to the isocenter 16 disposed on the rotation axis of the rotating gantry 28.

Returning to FIG. 1, the radiation therapy system 10 is equipped with stereoscopic imaging equipment including two pairs of an X-ray tube 23 and an X-ray detector 24, which is installed in the rotating gantry 28 such that two pairs mutually intersect by an angle of 90 degrees with respect to the isocenter 16. The X-ray detector 24 arranges a two-dimensional array of detection elements used to detect an X-ray 29.

An imaging unit 45 irradiates the X-ray 29 from the X-ray tube 23 to the isocenter 16. The X-ray detector 24 is configured to detect the X-ray 29 passing through the patient 25 via the detection elements based on an attenuation of energy such that the imaging unit 45 can receive a two-dimensional perspective image of the patient 25. The imaging unit 45 instructs a rotation control unit 48 to rotate the rotating gantry 28 in at least a quarter rotation.

The imaging unit 45 is configured to produce the second voxel data 12 upon acquiring a plurality of two-dimensional perspective images capturing the patient 25 in different directions. In this way, the imaging unit 45 captures the second voxel data 12 by rotating combinations of the X-ray tube 23 and the X-ray detector 24 about the isocenter 16 and in synchronization with the rotating gantry 28.

In this connection, the present embodiment exemplifies the integral structure including the X-ray tube 23 and the X-ray detector 24 integrally unified with the rotating gantry 28, but it is possible to adopt another structure and control which can rotate the X-ray tube 23 and the X-ray detector 24 without being synchronized with the rotating gantry 28. In this case, it is expected to increase the speed of the operation since the rotating gantry 28 as a large-size structure does not need to rotate by itself.

The identification unit 19b (19) is configured to identify the second position 22 of the focus 15b distinct from normal tissues in a coordinate system referring to an area having no positional displacement of patient's bones or a reference position of imaging equipment in the second voxel data 12. Therefore, the identification unit 19b (19) used in the treatment administration 14 has the same function as the identification unit 19a (19) used in the treatment planning 13. That is, it is possible to express both the first voxel data 11 and the second voxel data 12 in the common coordinate system.

The holding section 30 is configured to hold, as initial settings, the irradiation condition 35 of the beam 20 and the first movement information 31 of the tabletop 26, which are set in the treatment planning 13. Without changing the irradiation condition 35, the update unit 50 updates the first movement information 31 with the second movement information 32 such that the focal coverage rate 56 will be maximized at the focal position of the focus 15 of the patient 25. A setting unit 46 is configured to set the tabletop 26 for fixing the patient 25 thereon to the coordinate system of the treatment chamber 36 based on the updated second movement information 32.

After setting the tabletop 26 based on the second movement information 32, the rotation control unit 48 displaces the rotating gantry 28 in rotation based on the irradiation condition 35 set in the treatment planning 13. Then, an irradiation unit 47 irradiates the beam 20 to the patient 25 based on the irradiation condition 35.

The beam 20 is a type of radiation irradiated to focal tissues such as cancer to kill cells. As such a type of radiation, it is possible to mention X-rays, y-rays, electron beams, proton beams, and heavy particle beams. The present embodiment refers to the installation of the irradiation port 27 of the beam 20 installed in the rotating gantry 28 rotating about the isocenter 16, but this is not a limitation. For example, it is possible to adopt the fixation of the irradiation port 27 fixed to the treatment chamber 36.

For accurately positioning the tabletop 26 used to fix the patient 25 thereon, the imaging unit 45 for the second voxel data 12 may originally exemplify the usage of the function of capturing a two-dimensional perspective image to be cross-checked with the DDR (Digitally Reconstructed Radiograph) reconstructing the first voxel data 11 used in the treatment planning 13. However, this is not a limitation to the imaging unit 45 for the second voxel data 12. For example, the imaging unit 45 may be general-purpose medical stereoscopic imaging equipment, such as an X-ray CT or MRI equipment, which is installed separately at a position away from the isocenter 16 inside or outside of the treatment chamber 36.

### Second Embodiment

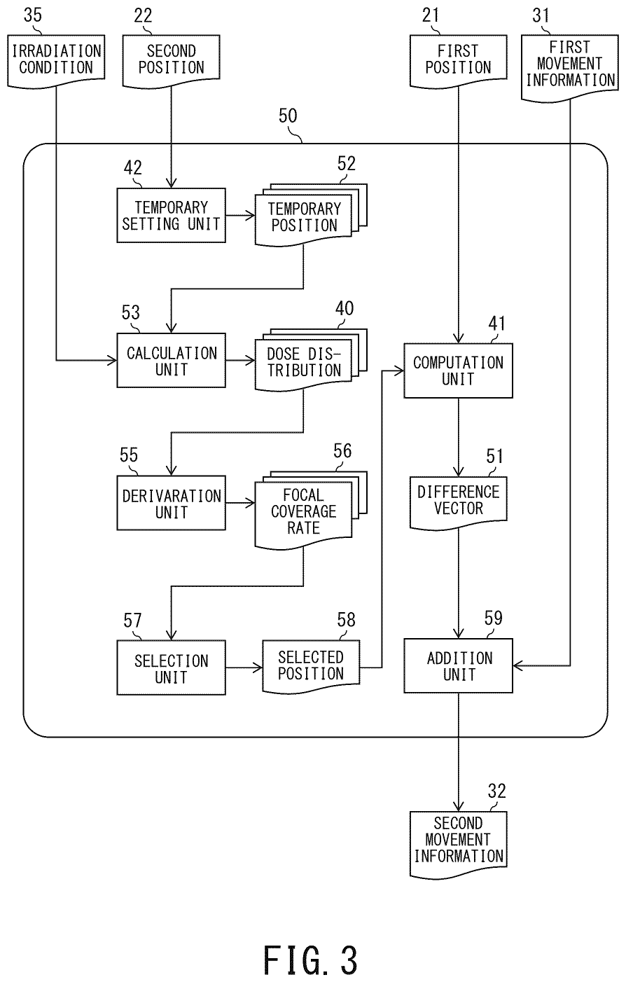

Next, the second embodiment of the present invention will be described with reference to FIG. 1 and FIG. 3. FIG. 3 is a configuration diagram of the update unit 50 of the radiation therapy system 10 according to the second embodiment. The radiation therapy system 10 of the second embodiment is characterized by the configuration of the update unit 50 among configurations in the first embodiment described above.

The update unit 50 in the second embodiment includes a calculation unit 53 configured to calculate a dose distribution 40 for the patient 25 when the beam 20 is irradiated to a plurality of temporary positions 52 in the peripheral region covering the second position 22 under the irradiation condition 35, a derivation unit 55 configured to derive the focal coverage rate 56 at the focal position of the focus 15 for a plurality of dose distributions 40, a selection unit 57 configured to select from among a plurality of temporary positions 52 a selected position 58 at which the focal coverage rate 56 has a maximum value, a computation unit 41 configured to produce a difference vector 51 representing a difference between the selected position 58 and the first position 21, and an addition unit 59 configured to add the difference vector 51 to the first movement information 31 and to thereby produce the second movement information 32.

A temporary setting unit 42 configured to temporarily set a plurality of temporary positions 52 assumed in the peripheral region covering the second position 22 in the coordinate system of the second voxel data 12. That is, the temporary setting unit 42 is configured to temporarily set a plurality of temporary positions 52 in the peripheral region covering the second position 22 identified by the second voxel data 12 according to the coordinate system of the second voxel data 12. Thus, it is possible to provide a plurality of temporary positions 52 about the second position 22 at regular intervals in the coordinate system of the second voxel data 12.

FIG. 4A is a graph showing a dose distribution 40a in the propagating direction of the beam 20 irradiated to the focus 15a at the first position 21 of the first voxel data 11 imaged in the stage of the treatment planning 13. FIG. 4B is a graph showing a dose distribution 40b in the propagating direction of the beam 20 irradiated to the focus 15b at the second position 22 of the second voxel data 12 imaged in the stage of the treatment administration 14. FIG. 4C is a graph showing a dose distribution 40c in the propagating direction of the beam 20 irradiated to the focus 15 at the selected position 58, which is selected such that the focal coverage rate 56 will have a maximum value by updating the position of the isocenter 16.

The calculation unit 53 is configured to calculate the dose distribution 40b (see FIG. 4B) in the interior body of the patient 25 when the beam 20 is irradiated to the isocenter 16 at the second position 22 under the irradiation conditions 35 set in the treatment planning 13. The derivation unit 55 is configured to derive a focal coverage rate 56b of the focus 15 for the dose distribution 40b.

Similarly, the calculation unit 53 is configured to calculate the dose distribution 40 in the interior body of the patient 25 when the beam 20 is irradiated to a plurality of temporary positions 52 under the irradiation condition 35 set in the treatment planning 13. The derivation unit 55 is configured to derive the focal coverage rate 56 of the focus 15 for a plurality of dose distributions 40. The selection unit 57 is configured to select from among a plurality of temporary positions 52 including the second position 22, the selected position 58 at which a focal coverage rate 56c (see FIG. 4C) has a maximum value.

Returning to FIG. 3, the computation unit 41 produces the difference vector 51 corresponding to the distance and the direction in which the focus 15 has been displaced in the first voxel data 11 or the second voxel data 12 during the period counted from the stage of the treatment planning 13 to the stage of the treatment administration 14. The addition unit 59 adds the difference vector 51 to the first movement information 31 to produce the second movement information 32 corresponding to the position of the focus 15 in the stage of the treatment administration 14 in the coordinate system of the treatment chamber 36.

The setting unit 46 (see FIG. 1) is configured to match the isocenter 16 with the selected position 58 of the focus 15 of the patient 25 by setting the position of the tabletop 26 based on the second movement information 32. In this connection, it is necessary to determine the validity of irradiating the beam 20 to the patient 25 with respect to the second movement information 32 updated in this way. It is possible to determine the validity based on a ratio of the focal coverage rate 56a (FIG. 4A), which is produced when the isocenter 16 is forced to match the first position 21, to the focal coverage rate 56c (FIG. 4C), which is produced when the isocenter 16 is forced to match the selected position 58. Specifically, it is ideal that the ratio between the focal coverage rate 56c at the selected position 58 and the focal coverage rate 56a at the first position 21 would be 100%.

The update unit 50 in the second embodiment is configured to update the first movement information 31 with the second movement information 32 by using the difference vector 51, but this is not a limitation. As another example of configuration, it is possible to think out a configuration for updating the first movement information 31 with the second movement information 32 by adopting optimization of hexa-degree freedom.

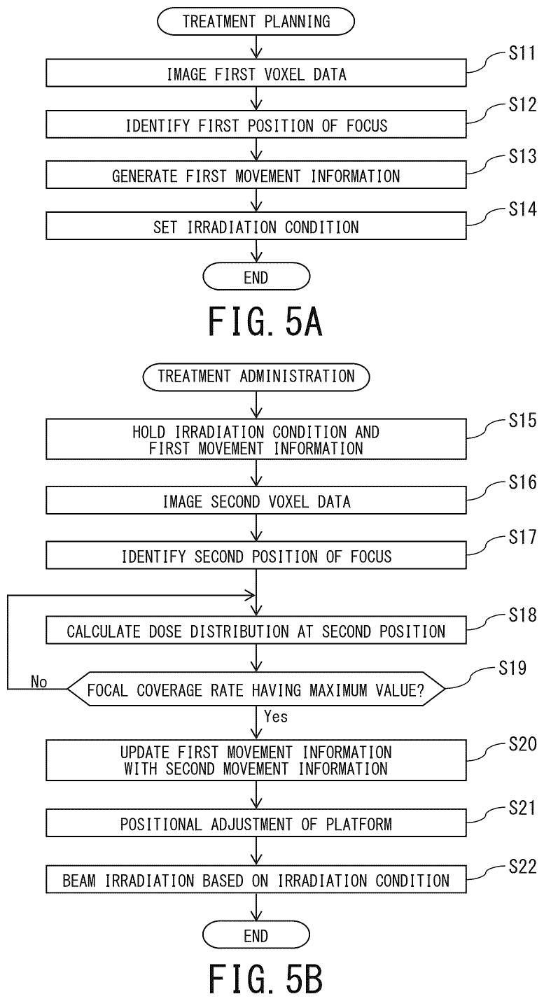

The procedure of a radiation therapy method and the algorithm of a radiation therapy program according to the present invention embodiment will be described with reference to the flowcharts of FIGS. 5A and 5B. FIG. 5A shows the procedure related to the treatment planning 13. In the stage of the treatment planning 13, the medical stereoscopic imaging equipment (e.g., X-ray CT or MRI equipment) is used to image the first voxel data 11 representative of the interior body of the patient 25 (S11). Subsequently, the first position 21 of the focus 15 is identified from the first voxel data 11 (S12).

Next, the first movement information 31 for moving the tabletop 26 is generated such that the isocenter 16 matches the first position 21 (S13). In addition, the irradiation condition 35 of the beam 20 is set such that the focal coverage rate 56a of the patient 25 irradiated with the beam 20 is maximized at the first position 21 (S14). Thus, the flow of FIG. 5A has ended.

FIG. 5B shows the procedure related to the treatment administration 14. In the stage of the treatment administration 14, the first movement information 31 and the irradiation condition 35 set in the treatment planning 13 are held (S15). Subsequently, the second voxel data 12 representing the interior body of the patient 25 is captured immediately before irradiating the beam 20 (S16). In addition, the second position 22 of the focus 15 is identified from the second voxel data 12 (S17).

Next, the dose distribution 40 at the position of the focus 15 of the patient 25 is calculated without changing the irradiation condition 35 (S18). When the focal coverage rate 56 at the position of the focus 15 according to the second movement information 32 indicates a maximum value (S19, Yes), the first movement information 31 is updated with the second movement information 32 (S20).

Next, the position of the tabletop 26 is adjusted based on the second movement information 32 (S21). Based on the irradiation condition 35, the beam 20 is irradiated to the patient 25 (S22). Thus, the flow in FIG. 5B has ended.

According to the radiation therapy system 10 of the foregoing embodiments in which the movement information of the tabletop 26 is updated to maximize the focal coverage rate 56 at the position of the focus 15 immediately before irradiating the beam 20, it is possible to maximize the absorbed dose of beams in focal tissues while keeping the absorbed dose of beams in normal tissues to a minimum, without changing the irradiation conditions 35, irrespective of any displacement of the position of the focus in the body occurring after the stage of the treatment planning 13. This may preclude the necessity of changing the irradiation condition 35 while reducing the time required to correct the irradiation condition 35, which may realize high-accuracy and short-time treatment, thus contributing to a reduction of burden on a patient and an improvement of treatment throughput.

Although several embodiments of the present invention have been described heretofore, the embodiments are illustrative and not intended to limit the scope of the invention. The embodiments can be implemented in various other forms, and therefore various omissions, substitutions, modifications, and combinations can be made without departing from the gist of the invention. The embodiments and modifications shall be included within the scope and the gist of the invention as well as within the scope of the invention as defined in claims and equivalents thereof.

The radiation therapy system described above includes a control device having a highly integrated processor such as a specified chip, a FPGA (Field Programmable Gate Array), a GPU (Graphics Processing Unit), or a CPU (Central Processing Unit), a storage device such as ROM (Read Only Memory) and RAM (Random Access Memory), an external storage device such as HDD (Hard Disk Drive) and SSD (Solid State Drive), a display device such as a display, an input device such as a mouse and a keyboard, and a communication interface (I/F), wherein the radiation therapy system can be realized by a hardware configuration using a generally-used computer. Therefore, the constituent elements of the radiation therapy system can be realized by a computer processor working on a radiation therapy program.

It is possible to provide the radiation therapy program incorporated in memory such as ROM in advance. Alternatively, it is possible to provide the radiation therapy program as files having the installable or executable format stored on a non-transitory computer-readable storage medium such as CD-ROM, CD-R, a memory card, DVD, and a flexible disk (FD).

The radiation therapy program according to the present embodiment may be stored on a computer connected to networks such as the Internet such that the radiation therapy program can be provided and downloaded through networks. The radiation therapy system can be established using individual modules capable of achieving the functions of constituent elements individually, which are combined and mutually connected to networks or private lines.

### REFERENCE SIGNS LIST

10 radiation therapy system
11 first voxel data
12 second voxel data
13 treatment planning
14 treatment administration
(15a, 15b) focus
16 isocenter
17 generation unit
18 adjustment unit
19 (19a, 19b) identification unit
20 beam
21 first position
22 second position
23 X-ray tube
24 X-ray detector
25 patient
26 tabletop
27 irradiation port
28 rotating gantry
29 X-ray
30 hold unit
31 first movement information
32 second movement information
35 irradiation conditions
36 treatment chamber
37 rotation drive unit
38 moving platform
40 (40a, 40b, 40c) dose distribution
41 computation unit
42 temporary setting unit
45 imaging unit
46 setting unit
47 irradiation unit
48 rotation control unit
50 update unit
51 difference vector
52 temporary position
53 calculation unit
55 derivation unit
56 (56a, 56b, 56c) focal coverage rate
57 selection unit
58 selected position
59 addition unit

1. A radiation therapy system comprising:
a holding unit configured to hold first movement information for moving a tabletop used to fix a patient's body thereon such that a first position of a patient's focus identified by first voxel data imaging an interior of a patient's body matches an isocenter in a dose of a beam administered to a patient's focus, and an irradiation condition, which is set to maximize a focal coverage rate of the patient's focus irradiated with the beam at the first position;
an identification unit configured to identify a second position of the patient's focus by second voxel data imaging the interior of the patient's body immediately before emitting the beam; and
an update unit configured to update the first movement information with second movement information to maximize the focal coverage rate at a position of the patient's focus without changing the irradiation condition.

2. The radiation therapy system according to claim 1, wherein the update unit comprises:
a calculation unit configured to calculate a dose distribution of a patient when the beam is emitted to a plurality of temporary positions in a peripheral region covering the second position under the irradiation condition;
a derivation unit configured to derive the focal coverage rate at the position of the patient's focus with respect to a plurality of dose distributions;
a selection unit configured to select one from among the plurality of temporary positions as a selected position at which the focal coverage rate has a maximum value;
a computation unit configured to compute a difference vector between the selected position and the first position; and
an addition unit configured to add the difference vector to the first movement information and to thereby output the second movement information, and
wherein the tabletop is moved such that the selected position matches the isocenter.

3. The radiation therapy system according to claim 2, wherein irradiating the beam to the patient is permitted based on a ratio of focal coverage rates to be derived when the isocenter matches the selected position and the first position.

4. The radiation therapy system according to claim 1, wherein the second voxel data is imaged by a combination of an X-ray tube and an X-ray detector that rotate together with an irradiation port of the beam about the isocenter in synchronization with a rotating gantry.

5. The radiation therapy system according to claim 1, wherein the second voxel data is imaged by equipment installed at a position deviated from the isocenter.

6. A radiation therapy method comprising:
holding first movement information for moving a tabletop used to fix a patient's body thereon such that a first position of a patient's focus identified by first voxel data imaging an interior of the patient's body matches an isocenter in a dose of a beam administered to the patient's focus;
holding an irradiation condition, which is set to maximize a focal coverage rate of the patient's focus irradiated with the beam at the first position;
identifying a second position of the patient's focus by second voxel data imaging the interior of the patient's body immediately before emitting the beam; and
updating the first movement information with second movement information to maximize the focal coverage rate at a position of the patient's focus without changing the irradiation condition.

7. A non-transitory computer-readable storage medium causing a computer to execute a radiation therapy program comprising the steps of:
holding first movement information for moving a tabletop used to fix a patient's body such that a first position of a patient's focus identified by first voxel data imaging an interior of the patient's body matches an isocenter in a dose of a beam administered to the patient's focus;
holding an irradiation condition, which is set to maximize a focal coverage rate of the patient's focus irradiated with the beam at the first position;
identifying a second position of the patient's focus by second voxel data imaging the interior of the patient's body immediately before emitting the beam; and
updating the first movement information with second movement information to maximize the focal coverage rate at a position of the patient's focus without changing the irradiation condition.

8. The radiation therapy system according to claim 2, wherein the second voxel data is imaged by a combination of an X-ray tube and an X-ray detector that rotate together with an irradiation port of the beam about the isocenter in synchronization with a rotating gantry.

9. The radiation therapy system according to claim 3, wherein the second voxel data is imaged by a combination of an X-ray tube and an X-ray detector that rotate together with an irradiation port of the beam about the isocenter in synchronization with a rotating gantry.

10. The radiation therapy system according to claim 2, wherein the second voxel data is imaged by equipment installed at a position deviated from the isocenter.

11. The radiation therapy system according to claim 3, wherein the second voxel data is imaged by equipment installed at a position deviated from the isocenter.
